(19) United States
(12) Patent Application Publication
(10) Pub. No.: US 2025/0171767 A1
(43) Pub. Date: May 29, 2025

(54) METHOD FOR SCREENING FUNCTIONAL NUCLEIC ACID APTAMERS TARGETING GPCR PROTEINS BY COMBINING CELL SORTING WITH FUNCTIONAL SORTING AND APPLICATION THEREOF

(71) Applicant: EYE HOSPITAL OF WENZHOU MEDICAL UNIVERS ITY, Wenzhou (CN)

(72) Inventors: Wencan WU, Wenzhou (CN); Yanchen ZHANG, Wenzhou (CN); Ende WU, Wenzhou (CN); Mao YE, Changsha (CN)

(73) Assignee: EYE HOSPITAL OF WENZHOU MEDICAL UNIVERS ITY, Wenzhou (CN)

(21) Appl. No.: 19/005,782

(22) Filed: Dec. 30, 2024

Related U.S. Application Data
(63) Continuation of application No. PCT/CN2024/112263, filed on Aug. 15, 2024.

(30) Foreign Application Priority Data
Sep. 25, 2023 (CN) 202311236620.0

(51) Int. Cl. C12N 15/10; G01N 33/50

(52) U.S. Cl. CPC C12N 15/1048; G01N 33/5023

(57) ABSTRACT

A method for screening functional nucleic acid aptamers targeting GPCR proteins by combining cell sorting with functional sorting and an and application thereof, wherein the native conformation of target proteins is maintained through cell sorting for target proteins, and the obtained nucleic acid aptamers have better in vivo specificity and targeting property than the nucleic acid aptamers obtained by conventional protein SELEX to construct Cell-SELEX of model cells. Compared with the conventional Cell-SELEX technology with unclear targets, the present disclosure has a stronger pertinence. The present disclosure can directly obtain functional aptamers that can affect the signaling pathway of target proteins and the cell phenotypes only by performing one round of functional sorting in cells after screening out a series of target aptamers at one time. It has the advantages of strong pertinence, high affinity, high cost performance, low manpower cost and broad-spectrum universality, etc.

### CROSS-REFERENCE TO RELATED APPLICATIONS

The application claims priority to Chinese patent application No. 202311236620.0, filed on Sep. 25, 2023, the entire contents of which are incorporated herein by reference.

### TECHNICAL FIELD

The present disclosure relates to the field of biotechnology, in particular to a method for screening functional nucleic acid aptamers targeting G protein-coupled receptor (GPCR) proteins by combining cell sorting with functional sorting and an application thereof.

### BACKGROUND

GPCR proteins are a large family of membrane protein receptors, which play a central role in the signal transduction process and are responsible for important physiological functions of cells. More than half of the drugs currently on the market are developed for GPCR. However, as a membrane protein, GPCR has a large molecular weight and is in a complex molecular structure, so it is difficult to synthesize and purify GPCRs and maintain their native conformation without a lipid bilayer structure. Due to the inherent technical barriers of GPCR proteins, there are limited methods for studying small molecule GPCR drugs. And an aptamer specifically recognizes targets with complementary shapes, so it is very important to select and prepare selective targets with a native conformation.

Nucleic acid aptamers are single-stranded oligonucleotides that are typically obtained through a systematic evolution of ligands by exponential enrichment (SELEX), have unique tertiary conformations and interact with target topologies with relatively high shape complementarities. One of the most important features of SELEX or aptamers is the ability to generate aptamers for various targets with high affinity and specificity, from small molecules to proteins, bacteria and cells, regardless of the complexity and molecular weight of the target. Compared with antibodies, aptamers are simple in synthesis and modification and have the advantages of low molecular weight, low immunogenicity, good tissue permeability and recognition. Currently, in the process of translating basic research into clinical trials, various aptamers have become therapeutic agents, such as pegaptanib, E10030, ARC1779, AS1411, NOXA12, and NOX-H94.

GPCR protein, also known as G-coupled protein, is a cell membrane protein. At present, 40% of the targets developed for FDA-approved drug species are GPCR proteins. As a second messenger, cAMP plays an important role in signal transduction. The cell signaling of G protein-coupled receptors (GPCRs) can be assessed by testing downstream effectors. Detecting the level of cAMP, the second messenger produced by adenylate cyclase activation, is one of the most common methods to screen GPCR agonists and antagonists. Hormone→GPCR→G protein→adenylate cyclase→cAMP→CAMP-dependent protein kinase A→gene regulatory protein→gene transcription. Therefore, it is of great significance to screen specific receptor agonists or antagonists with high activity and long half-life by using GPCR as a drug screening target. However, due to the chemical properties of GPCRs and the scarcity of native samples, there are inherent technical barriers in the development of novel compounds targeting GPCRs to efficiently express and purify proteins in their native conformations. Due to the inherent technical barriers of GPCR proteins, there are few methods for studying small molecule inhibitor targets and few reports on GPCR-targeted aptamers, and all reported small-molecule targeted Thyroid stimulating hormone receptors (TSHRs) are still in the preclinical stage. Since the extracellular domain of a GPCR is limited as an epitope and it is not possible to maintain its native conformation in the absence of a lipid bilayer, generating molecules that recognize the native GPCR conformation is challenging. In some previous GPCR studies, recombinant GPCR proteins have been used, such as NTSR1, B2-adrenergic receptor and CCR5, whose overexpression, purification and recombination are laborious and time-consuming.

### SUMMARY

In order to address the technical defects existing in the prior art, the present disclosure provides a method for screening functional nucleic acid aptamers targeting GPCR proteins by combining cell sorting with functional sorting and an application thereof. The SELEX technology for nucleic acid aptamers targeting GPCR proteins by combining cell sorting with functional sorting can screen out specific nucleic acid aptamers while maintaining the native activity of proteins, and can screen out functional nucleic acid aptamers affecting GPCR signal pathways combined with further functional sorting.

The technical solution proposed in the present disclosure is: a method for screening functional nucleic acid aptamers targeting GPCR proteins by combining cell sorting and functional sorting, comprising the following steps:
(1) constructing and validating stable model cells selected by a cell sorting;
(2) constructing a screening library;
(3) enriching the screening library going through a cell sorting to obtain an enriched library;
(4) sequencing the enriched library after the cell sorting;
(5) performing a family analysis and a percentage analysis of aptamer candidates;
(6) performing an affinity and specificity analysis of aptamer candidates: including carrying out a flow cytometry screening to obtain the target affinity of an aptamer; performing a Pull-down experiment to verify the target protein of the aptamer; performing a target protein knockdown experiment to verify the target protein of the aptamer;
verifying the target protein of the aptamer with tissue sections and the localization of antibodies.
(7) performing a functional sorting of aptamer candidates for the second messenger cAMP; and
(8) performing a functional detection of cell phenotypes by sorting functional aptamers.

The constructing model cells in the step (1) is as follows:
(a) constructing protein expression plasmids and empty plasmids: according to the sequence of a gene database, designing PCR primers according to principles of the primer design, amplifying a full-length CDS sequence of the target gene, cloning the PCR products into plasmids after a double enzymatic cleavage, and constructing the target protein expression plasmids and empty plasmids without a target gene expression; wherein the 5′- and 3′-ends of the PCR products carry sequences homologous to plasmids;
(b) lentivirus packaging: co-transfecting the above-mentioned two plasmids and a helper plasmid into HEK293T cells respectively by a calcium phosphate/DNA precipitation method to produce high-titer lentiviruses and complete the lentivirus packaging;
(c) virus concentration: collecting a supernatant of HEK293T cells, filtering it after removing debris by centrifugation, filtering the culture medium into a collection bottle thereunder, discarding most of the culture medium in the collection bottle, then centrifuging the viruses into the culture medium with the filter placed upside down, with viruses then being concentrated;
(d) construction of cell strains stably expressing target proteins: inoculating HEK293T cells with two concentrated lentiviruses, infecting them for 48 h, screening them with a culture medium containing antibiotics, collecting cells and amplifying them to obtain cell strains stably expressing target proteins and cell strains without target protein expression; and
(e) validation of expression of target proteins: in terms of cell strains stably expressing target proteins and cell strains without target protein expression, validating the expression of target proteins by a flow cytometry test, an immunofluorescence test and a Western Blot test.

The obtaining an enriched library by cell sorting and enriching in the step (3) is as follows:
(i) after denaturation of an original DNA library at 95° C. followed by renaturation treatment at 4° C., performing a positive screening of HEK-293 Ts with high expression of target proteins, as positive screening cells, to obtain a positively screened supernatant and a positively screened cell sap;
(ii) after denaturation and renaturation of the positively screened cell sap, performing a negative screening of cell strains without target protein expression, as negative screening cells, to obtain a negatively screened supernatant and a negatively screened cell sap;
(iii) positively screening the negatively screened supernatant and the positive screening cells to obtain a positively screened supernatant and a positively screened cell sap;
(iv) performing a PCR test on the negatively screened supernatant and the positively screened cell sap, and obtaining products obtained in this round of screening according to the PCR results;
(v) recycling the products after PCR amplification for the next round of screening after denaturation and desalting, detecting the process of screening library by a flow cytometry, and obtaining an enriched library by cell sorting according to the results of the flow cytometry.

The functional sorting in the step (7) includes:
(i) after incubating several aptamer candidates with positive screening cells respectively and adding cyclic nucleotide phosphodiesterase inhibitor (IBMX), measuring the intracellular or cell supernatant cAMP content by an enzyme-linked immunosorbent assay (ELISA);
(ii) screening out the aptamers that affect cAMP production of GPCR signaling pathway, and exploring their effects on cell phenotypes by cell phenotypic experiments; and
(iii) exploring the aptamers' effect on signaling pathways by Western Blot.

An application of the functional nucleic acid aptamer targeting GPCR proteins obtained by the method in preparing a reagent, kit or imaging agent for specifically recognizing and quantifying GPCR proteins is provided.

An application of the functional nucleic acid aptamer targeting GPCR proteins obtained by the method in preparing an agonist or inhibitor of GPCR protein molecules is provided.

An application of the functional nucleic acid aptamer targeting GPCR proteins obtained by the method in preparing a drug for preventing and/or treating and/or reversing GPCR-related diseases is provided.

The present disclosure has the following beneficial effects: The present disclosure provides a method for screening functional nucleic acid aptamers targeting GPCR proteins by combining cell sorting with functional sorting and an and application thereof, wherein the native conformation of target proteins is maintained through cell sorting for target proteins, and the obtained nucleic acid aptamers have better in vivo specificity and targeting property than the nucleic acid aptamers obtained by conventional protein SELEX to construct Cell-SELEX of model cells. Compared with the conventional Cell-SELEX technology with unclear targets, the present disclosure has a stronger pertinence, and can obtain a series of target-specific aptamers through one screening as a library for functional sorting of cells. Compared with the conventional screening method that requires exploring whether an aptamer has functions in the cell after screening, the present disclosure can directly obtain functional aptamers that can affect the signaling pathway of target proteins and the cell phenotypes only by performing one round of functional sorting in cells after screening out a series of target aptamers at one time. It has the advantages of strong pertinence, high affinity, high cost performance, low manpower cost and broad-spectrum universality, etc.

### BRIEF DESCRIPTION OF DRAWINGS

FIG. 1 is a schematic diagram of the model cell construction.

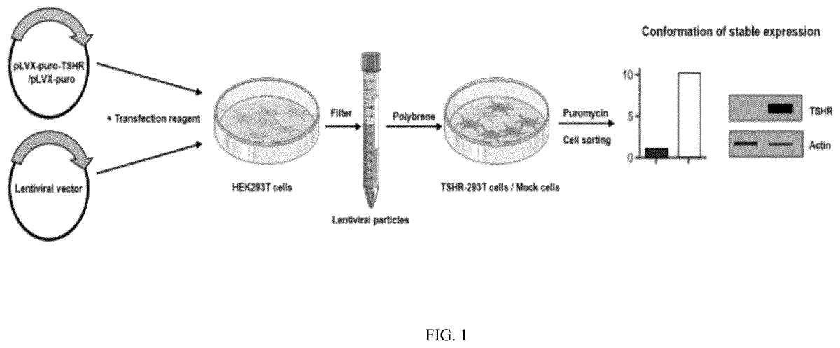

FIG. 2 shows validating the construction of model cells by a flow cytometry test.

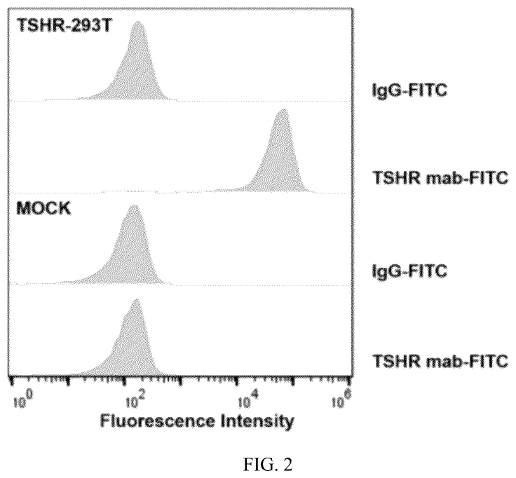

FIG. 3 shows validating the construction of model cells by an immunofluorescence test.

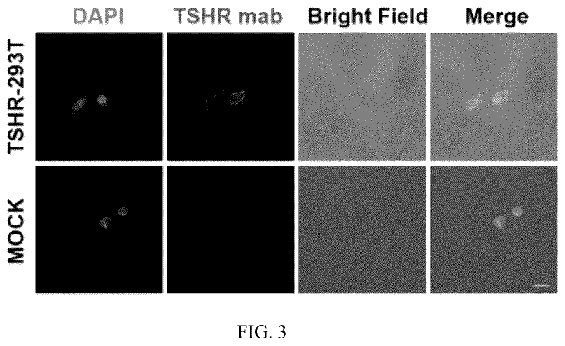

FIG. 4 shows validating the construction of model cells by a Western Blot test.

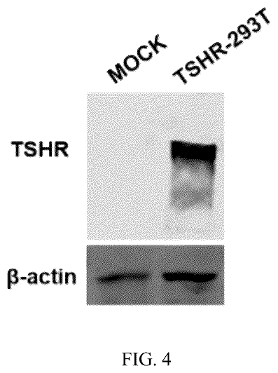

FIG. 5 shows the affinities of aptamer libraries at different rounds to positive and negative cells.

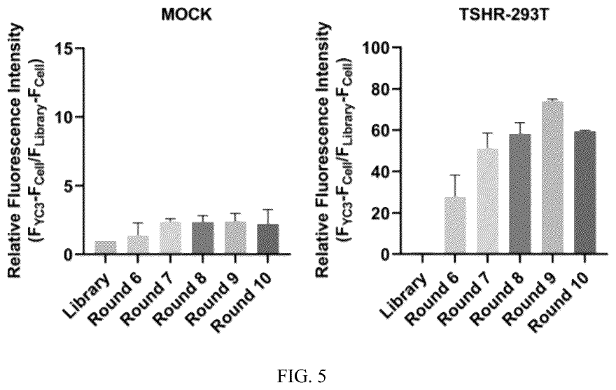

FIG. 6 shows the affinities of a series of aptamer candidates to positive and negative cells.

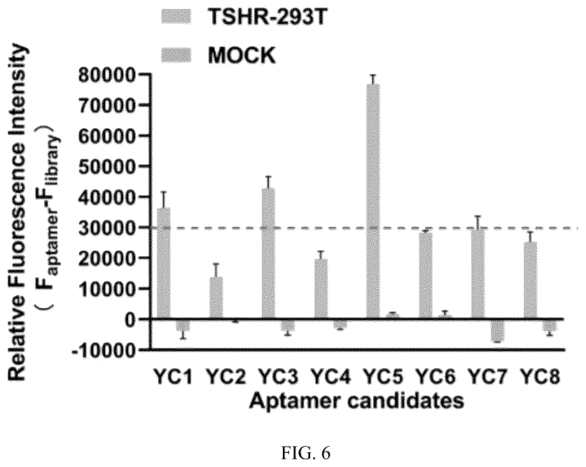

FIG. 7 shows the effect of aptamer candidates on the CAMP content in TSHR-293T cells as determined by an ELISA.

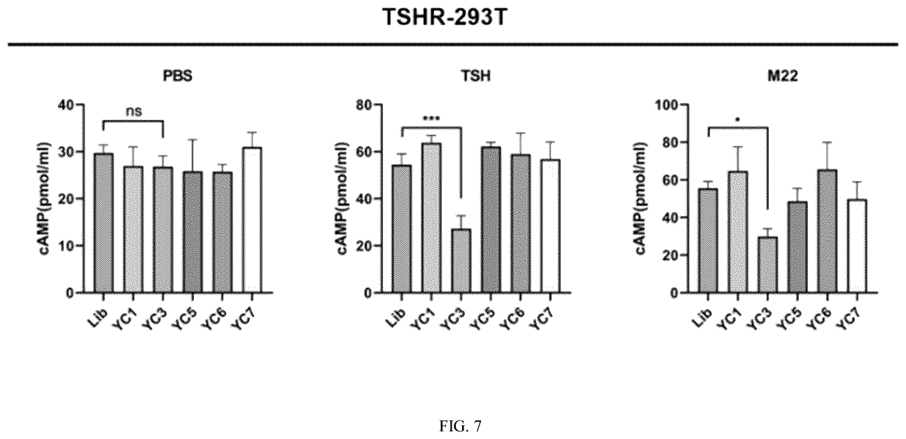

FIG. 8 shows the effect of nucleic acid aptamer YC3 on cytokines IL-6 and IL-8 determined by an ELISA.

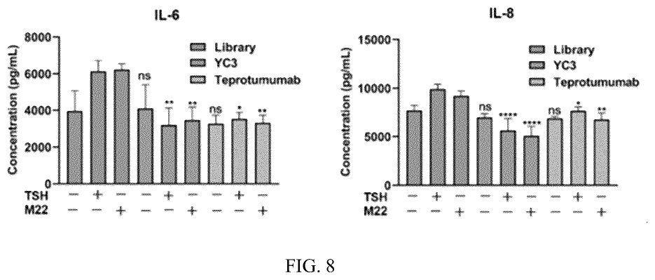

### DETAILED DESCRIPTION OF THE EMBODIMENTS

The technical solutions in the embodiments of the present disclosure will be clearly and completely described below with reference to the drawings in the embodiments of the present disclosure. Obviously, the embodiments described are only a part of, not all, the embodiments of the present disclosure. Based on the embodiments described herein, all other embodiments obtained by those of ordinary skill in the art without creative work are within the scope of the present disclosure.

Unless otherwise specified, the experimental materials used in the following embodiments are purchased from conventional biochemical reagent stores.

Cell source: Primary fibroblasts (GOFs) extracted from adipose connective tissue isolated from patients with thyroid-related diseases during orbital decompression were used in this experiment; the human embryonic kidney cell line (293T) was purchased from Wuhan Pricella Biotechnology Co., Ltd. in China.

Take the TSHR protein in GPCR as an example

### Embodiment 1

Construct and validate the stable model cells obtained by cell sorting, i.e. positive screening cells with high expression of TSHR plasmids stably transfected by lentiviruses and negative screening cells with empty plasmids stably transfected by lentiviruses.

Construction of protein expression plasmids and empty plasmids

According to the sequence of NCBI gene database, the software Primer 5 is used to design PCR primers (primer information is shown in Table 1) according to principles of the primer design. The full-length sequence of hTSHR CDS of amplified target gene is prepared and the PCR reaction system is prepared: The template, primer, dNTP, enzyme, reaction buffer and ddH2O are added in proportion, slightly dissociated, and put into a PCR instrument (C1000, Thermal Cycle, BIO-RAD) to amplify the target fragment, and the PCR products are cloned into plasmids after a double enzymatic cleavage with a seamless cloning technology to construct the TSHR expression plasmids and empty plasmids without a target gene expression; wherein the 5′- and 3′-ends of the PCR products carry sequences homologous to plasmids;

TABLE 1
PCR primers and their sequences

| Primer name | Sequence (5′-3′) |
|---|---|
| Forward | GGAATTCATGAGGCCGGCGGACTTGCT |
| Reverse | CGGGATCCTTACAAAACCGTTTGCATAT |

### Lentivirus Packaging

Co-transfect the above-mentioned two plasmids and a helper plasmid into HEK293T cells respectively by a calcium phosphate/DNA precipitation method (clontech kit) to produce high-titer lentiviruses and complete the lentivirus packaging;

HEK293T cells are passaged 24 h before transfection and can be transfected when the cell density reaches 80%.

Add choloroquine with a final concentration of 15 ul 50 mM to a fresh culture medium 3 h before transfection. Reagents required for transfection by swirling: Prepare two centrifuge tubes for transfection samples in each 10 cm culture dish, and add the following reagents in sequence:

| Solution A | Plasmid | 3 | μg |
|---|---|---|---|
| | pack A plasmid | 9 | μg |
| | pack B plasmid | 3 | μg |
| | pack C plasmid | 3 | μg |
| | Sterile H₂0 | To 700 μL | |
| | 2M Calcium Solution | 87 | μL |
| Solution B | 2X HBS | 700 | μL |

Gradually add solution B while swirling gently solution A. After completion, the solution becomes opaque due to precipitation. Incubate for 15 min at room temperature. Swirl evenly again the transfection complex before adding it to cells. Gently add the precipitate to the culture plate drop by drop and in multiple positions, and shake the culture plate back and forth after complete addition so that the precipitate is evenly adsorbed on the cell surface. Change the medium after 8 h of transfection.

### Virus Concentration

Collect the supernatant of the above-mentioned HEK293T cells, centrifuge it to remove debris and filter it with a 0.45 μm filter. The virus cannot be filtered and remains above the filter, and the culture medium is filtered into a collection bottle thereunder. Discard most of the culture medium in the collection bottle, then centrifuge the viruses into the culture medium with the filter placed upside down, with viruses then being concentrated.

### Construction of Cell Strains Stably Expressing Target Proteins

Inoculate HEK293T cells with two concentrated lentiviruses and 4 μg/mL polybrene, infect them for 48 h, screen them with a culture medium containing puromycin antibiotics for 3 days, collect cells and amplify them to obtain cell strains stably expressing target proteins and cell strains without target protein expression.

### Validation of Expression of Target Proteins

In terms of cell strains stably expressing target proteins and cell strains without target protein expression, validate the expression of target proteins by a flow cytometry test, an immunofluorescence test and a Western Blot test.

### Flow Cytometry

Culture two model cells for more than 48 h to make the cell density reach 90%, and then digest adherent cells from the culture dish with 0.2% EDTA, respectively. Incubate the obtained cells separately with FITC-marked IgG2a (Bioss, China) for 30 min at room temperature and then with anti-TSHR antibodies (ab27974, Abcam) for 1 h at room temperature. After 3 washes, incubate cells with FITC-marked secondary antibodies for 30 min at room temperature and then wash them 3 times for a flow cytometry assay.

### Immunofluorescence Test

Implant cells into the bottom of an optical culture dish and incubate them for 24 h. For aptamers, fix cells with 4% paraformaldehyde for 10 min at room temperature after washing them with D-PBS. After washing with D-PBS, block cells in 5% BSA/DPBS buffer for 1 h and then incubate them overnight at 4° C. with anti-TSHR antibodies (1:500, Abcam, ab27974). After completion of the washing step, stain nuclei with secondary antibodies for 1 h at room temperature and then with DAPI for 10 min. Use Cell Observer SD (Leica, Bannockburn, IL, USA) to acquire images.

### Western Blot

Rinse cultured cells with cold D-PBS and lyse them in RIPA lysis buffer using a protease inhibitor cocktail (EpiZyme). Use protein detection reagents (EpiZyme) to determine protein concentration. Electrophorese a protein sample (40 μg/well) with 10% SDS-PAGE and add it to protein loading buffer. Transfer proteins then to 0.22-μm NC membranes using a Western blot transfer system (Bio-Rad) at 300 mA for 90 min. Wash NC membranes with TBST (0.5% Tween 20 in Tris-buffered saline) and then block them with 5% nonfat milk for 1 h at room temperature. After that, wash the membranes three times with TBST and incubate them overnight at 4° C. with anti-TSHR antibodies (1:1000, Abcam, ab27974) and anti-β-actin antibodies (1:1000, Proteintech, 66009-1-Ig). After washing three times with TBST, incubate them for 1 h at room temperature with goat anti-mouse IgG (heavy & light chain) antibodies (1:50 000, LI-COR, IRDye680CW), then detect them by Odyssey LI-COR.

### Embodiment 2: Nucleic Acid Aptamer Screening for Target Proteins

### Design of Nucleic Acid Library and Primers:

Random single-stranded DNA library (Not only
listed):
5′-ACCGACCGTGCTGGACTCA(N)42ACTATGAGCGAGCCTGGCG-3′
(N represents any of the A, T, G and C bases)
Upstream primer:
5′-FITC-ACCGACCGTGCTGGACTCA-3
Downstream primer:
5′-biotin-CGCCAGGCTCGCTCATAGT-3

### Screening Process:

In the present disclosure, TSHR-293 Ts with high expression of TSHR are used as positive screening targets, and MOCK cells without expression of TSHR are used as negative screening targets.

### Positive Screening:

a. Incubation: Dissolve the above random DNA library with binding buffer, denature it at 95° C. for 5 min and renature it on ice for 10 min, then co-incubate it at 4° C. for 1 h with pretreated TSHR-293T whose cell confluency reached about 90% after more than 48 h of culture.

b. Separation: Remove the supernatant after incubation, rinse the incubated cells with washing buffer several times, then scrape the washed cells and centrifuge tubes with sterile water, perform denaturation at 95° C. for 10 min, renaturation on ice for 10 min, and centrifugation at 5500 rpm for 3 min; aspirate the supernatant to isolate the first round of screening nucleic acid library of TSHR-293T cells.

c. PCR amplification library: Use the library obtained in step b as a template, and use the above primers as primers. The amplification conditions are as follows: 95° C., 30 s; 55.9° C., 30 s; 72° C., 30 s, 8 cycles of amplification, 72° C., 5 min. After obtaining the preliminary amplification product, amplify for an appropriate number of cycles with the amplification product as a template for mass amplification.

d. Preparation of single strands of DNA: Separate the antisense strand of PCR amplification product in step c labeled with biotin by agarose beads modified with streptavidin, denature double-stranded DNA with 0.2 M NaOH, and collect the sense single-stranded DNA library labeled with fluorescein isothiocyanate by desalting.

Negative screening: Incubate the single-stranded DNA library obtained in step d with negative screening MOCK cells, collect the supernatant after incubation to exclude non-specifically bound nucleic acid molecules, and continue to incubate them with positive screening cells for the next screening step.

Cycle of screening process: Repeat the screening processes of to until a nucleic acid aptamer library with strong binding to target cells' TSHR-293T cells is screened. This process needs to be repeated for several to more than a dozen times.

High-throughput sequencing: The nucleic acid library with the largest binding in the last round of screening will be subjected to high-throughput sequencing, and the binding ability between the obtained sequences and TSHR-293T cells will be detected by a flow cytometry test, so as to determine nucleic acid aptamers. Perform a fluorescence detection by a flow cytometry test, with the initial random DNA library served as a control.

### Embodiment 3: Screening Functional Aptamer Chains

Screening of Aptamers Influencing Protein Signaling Pathways by cAMP Content in Cell Supernatants Determined by an ELISA

Inoculate cells in 24-well plates and starve them overnight with 1% fetal bovine serum. The following day, incubate cells for 30 min in serum-free antibiotic-free medium containing 0.5 mM cAMP phosphodiesterase inhibitor 3-isobutyl-1-methylxanthie (IBMX) (MedChemExpress, USA), to avoid cAMP degradation, then add TSH (100 ng/mL), M22 (100 ng/ml) or the nucleic acid aptamer candidate for 6 h. Collect and store cell supernatants at −80° C. until the CAMP assay (KGE002B; R&D Systems, Minneapolis, Minnesota) that uses polyclonal antibodies to competitively bind standard or sample supernatants of cAMP. All measurements of CAMP release are performed twice as previously described.

### Effect of Aptamers on Cell Phenotypes

Inoculate GOFs in 24-well plates and incubate them overnight with 1% FBS. The following day, treat cells with or without TSH (100 ng/mL), M22 (100 ng/mL), aptamer YC3, or Teprotumumab in DMEM supplemented with 1% FBS. After 48 h, take the supernatant of cell culture by centrifugation at 5000 g for 10 min and store it at −80° C. for later use. Measure IL-6 and IL-8 contents with a commercial ELISA kit (FineTest, Wuhan, China), following the manufacturer's instructions. Dilute samples 1:10 prior to analysis and analyze for three times. Detection of effect of aptamers on cellular pathways by a Western Blot test

Rinse cultured cells with cold D-PBS, lyse them in RIPA lysis buffer using a protease inhibitor cocktail (EpiZyme), and add protease and phosphatase inhibitors (EpiZyme). Use protein detection reagents (EpiZyme) to determine protein concentration. Add a protein sample (40 μg/well) to a protein loading buffer, boil it at 95° C. for 5 min, and electrophorese it in 10% SDS-PAGE. Transfer proteins then to 0.22-μm NC membranes using a Western blot transfer system (Bio-Rad) at 300 mA for 90 min. Wash NC membranes with TBST (0.5% Tween 20 in Tris-buffered saline) and then block them with 5% nonfat milk for 1 h at room temperature. After that, wash membranes three times with TBST and incubate primary and anti-actin antibodies (1:1000, Proteintech, 66009-1-Ig) overnight at 4° C. After washing three times with TBST, incubate them for 1 h at room temperature with secondary antibodies (1:50 000, LI-COR) and then detect them by Odyssey LI-COR.

People skilled in the art should know that: although the present disclosure has been described according to the above specific embodiments, the inventive idea of the present disclosure is not limited to this disclosure. Any modification using the inventive idea will be covered by the protection scope of this disclosure.

The above are only preferred embodiments of the present disclosure, and the protection scope of the present disclosure is not limited to the above embodiments. All technical solutions under the idea of the present disclosure shall fall within the protection scope of the present disclosure. It should be pointed out that for ordinary technical personnel in this field, several improvements and modifications without departing from the principle of the disclosure shall also be deemed as the protection scope of the disclosure.

What is claimed is:

1. A method for screening functional nucleic acid aptamers targeting GPCR proteins by combining cell sorting with functional sorting, comprising the following steps:
(1) constructing and validating stable model cells selected by a cell sorting;
(2) constructing a screening library;
(3) enriching the screening library going through a cell sorting to obtain an enriched library;
(4) sequencing the enriched library after the cell sorting;
(5) performing a family analysis and a percentage analysis of aptamer candidates;
(6) performing an affinity and specificity analysis of aptamer candidates: including carrying out a flow cytometry screening to obtain the target affinity of an aptamer; performing a Pull-down experiment to verify the target protein of the aptamer; performing a target protein knockdown experiment to verify the target protein of the aptamer;
verifying the target protein of the aptamer with tissue sections and the localization of antibodies;
(7) performing a functional sorting of aptamer candidates for the second messenger cAMP; and
(8) performing a functional detection of cell phenotypes by sorting functional aptamers.

2. The method according to claim 1, wherein the constructing model cells in the step (1) is as follows:
(a) constructing protein expression plasmids and empty plasmids: according to the sequence of a gene database, designing PCR primers according to principles of the primer design, amplifying a full-length CDS sequence of the target gene, cloning the PCR products into plasmids after a double enzymatic cleavage, and constructing the target protein expression plasmids and empty plasmids without a target gene expression; wherein the 5′- and 3′-ends of the PCR products carry sequences homologous to plasmids;
(b) lentivirus packaging: co-transfecting the above-mentioned two plasmids and a helper plasmid into HEK293T cells respectively by a calcium phosphate/DNA precipitation method to produce high-titer lentiviruses and complete the lentivirus packaging;
(c) virus concentration: collecting a supernatant of HEK293T cells, filtering it after removing debris by centrifugation, filtering the culture medium into a collection bottle thereunder, discarding most of the culture medium in the collection bottle, then centrifuging the viruses into the culture medium with the filter placed upside down, with viruses then being concentrated;
(d) construction of cell strains stably expressing target proteins: inoculating HEK293T cells with two concentrated lentiviruses, infecting them for 48 h, screening them with a culture medium containing antibiotics, collecting cells and amplifying them to obtain cell strains stably expressing target proteins and cell strains without target protein expression; and
(e) validation of expression of target proteins: in terms of cell strains stably expressing target proteins and cell strains without target protein expression, validating the expression of target proteins by a flow cytometry test, an immunofluorescence test and a Western Blot test.

3. The method according to claim 1, wherein the obtaining an enriched library by cell sorting and enriching in the step (3) is as follows:
(i) after denaturation of an original DNA library at 95° C. followed by renaturation treatment at 4° C., performing a positive screening of HEK-293 Ts with high expression of target proteins, as positive screening cells, to obtain a positively screened supernatant and a positively screened cell sap;
(ii) after denaturation and renaturation of the positively screened cell sap, performing a negative screening of cell strains without target protein expression, as negative screening cells, to obtain a negatively screened supernatant and a negatively screened cell sap;
(iii) positively screening the negatively screened supernatant and the positive screening cells to obtain a positively screened supernatant and a positively screened cell sap;
(iv) performing a PCR test on the negatively screened supernatant and the positively screened cell sap, and obtaining products obtained in this round of screening according to the PCR results;
(v) recycling the products after PCR amplification for the next round of screening after denaturation and desalting, detecting the process of screening library by a flow cytometry, and obtaining an enriched library by cell sorting according to the results of the flow cytometry.

4. The method according to claim 1, wherein the functional sorting in the step (7) comprises:
(i) after incubating several aptamer candidates with positive screening cells respectively and adding cyclic nucleotide phosphodiesterase inhibitor (IBMX), measuring the intracellular or cell supernatant cAMP content by an enzyme-linked immunosorbent assay (ELISA);
(ii) screening out the aptamers that affect cAMP production of GPCR signaling pathway, and exploring their effects on cell phenotypes by cell phenotypic experiments; and
(iii) exploring the aptamers' effect on signaling pathways by Western Blot, such as p-ERK, p-MEK, AKT expression, NF-kappaB-p65 nuclear translocation.

5. An application of the functional nucleic acid aptamer targeting GPCR proteins obtained by the method according to claim 1 in preparing a reagent, kit or imaging agent for specifically recognizing and quantifying GPCR proteins.

6. An application of the functional nucleic acid aptamer targeting GPCR proteins obtained by the method according to claim 1 in preparing an agonist or inhibitor of GPCR protein molecules.

7. An application of the functional nucleic acid aptamer targeting GPCR proteins obtained by the method according to claim 1 in preparing a drug for preventing and/or treating and/or reversing GPCR-related diseases.
